(19) United States
(12) Patent Application Publication
(10) Pub. No.: US 2024/0255842 A1
(43) Pub. Date: Aug. 1, 2024

(54) HARDMASK COMPOSITION, HARDMASK LAYER AND METHOD OF FORMING PATTERNS

(71) Applicant: SAMSUNG SDI CO., LTD., Yongin-si (KR)

(72) Inventors: Sangmi KIM, Suwon-si (KR); Sunghwan KIM, Suwon-si (KR)

(21) Appl. No.: 18/411,690

(22) Filed: Jan. 12, 2024

(30) Foreign Application Priority Data
Jan. 17, 2023 (KR) 10-2023-0006893

(51) Int. Cl. G03F 1/68

(52) U.S. Cl. CPC G03F 1/68

(57) ABSTRACT

A hardmask composition, a hardmask layer including a cured product of the aforementioned hardmask composition, and a method of forming patterns that includes using the hardmask layer including a cured product of the aforementioned hardmask composition, the hardmask composition including a solvent and a compound represented by Chemical Formula 1:

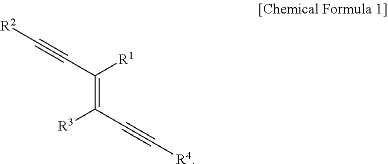

### CROSS-REFERENCE TO RELATED APPLICATION

This application claims priority to and the benefit of Korean Patent Application No. 10-2023-0006893, filed in the Korean Intellectual Property Office on Jan. 17, 2023, the entire contents of which are incorporated herein by reference.

### BACKGROUND

### 1. Field

Embodiments are directed to a hardmask composition, a hardmask layer including a cured product of the hardmask composition, and a method of forming patterns using the hardmask composition.

### 2. Description of the Related Art

Recently, the semiconductor industry has developed an ultra-fine technique having a pattern of several to several tens nanometer size. Such an ultrafine technique needs effective lithographic techniques.

### SUMMARY

The embodiments may be realized by providing a hardmask composition including a solvent and a compound represented by Chemical Formula 1:

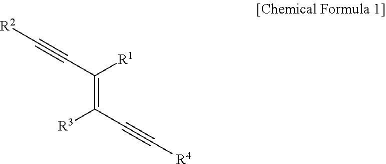

in Chemical Formula 1, R¹ and R³ are each independently a substituted or unsubstituted C6 to C30 aromatic hydrocarbon group, a substituted or unsubstituted C3 to C30 heteroaromatic ring group, or a combination thereof, R² and R⁴ are each independently a substituted or unsubstituted C6 to C30 aromatic hydrocarbon group, a substituted or unsubstituted C3 to C30 heteroaromatic ring group, a substituted or unsubstituted monovalent C3 to C30 saturated or unsaturated alicyclic hydrocarbon group, or a combination thereof.

The embodiments may be realized by providing a hardmask layer including a cured product of the hardmask composition.

The embodiments may be realized by providing a method of forming patterns using the hardmask composition.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

As used herein, when a definition is not otherwise provided, ‘substituted’ may refer to replacement of a hydrogen atom of a compound by a substituent selected from a halogen atom (F, Br, Cl, or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a vinyl group, a C1 to C20 alkyl group, a C2 to C20 alkenyl group, a C2 to C20 alkynyl group, a C6 to C30 aryl group, a C7 to C30 arylalkyl group, a C9 to C30 allylaryl group, a C1 to C30 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, or a combination thereof.

In addition, adjacent two substituents of the substituted halogen atom (F, Br, Cl, or I), the hydroxy group, the nitro group, the cyano group, the amino group, the azido group, the amidino group, the hydrazino group, the hydrazono group, the carbonyl group, the carbamyl group, the thiol group, the ester group, the carboxyl group or the salt thereof, the sulfonic acid group or the salt thereof, the phosphoric acid or the salt thereof, the C1 to C30 alkyl group, the C2 to C30 alkenyl group, the C2 to C30 alkynyl group, the C6 to C30 aryl group, the C7 to C30 arylalkyl group, the C1 to C30 alkoxy group, the C1 to C20 heteroalkyl group, the C3 to C20 heteroarylalkyl group, the C3 to C30 cycloalkyl group, the C3 to C15 cycloalkenyl group, the C6 to C15 cycloalkynyl group, the C2 to C30 heterocyclic group may be fused to form a ring.

As used herein, when a definition is not otherwise provided, “aromatic hydrocarbon” refers to a group having one or more hydrocarbon aromatic moieties, including a form in which hydrocarbon aromatic moieties are linked by a single bond, a non-aromatic fused ring form in which hydrocarbon aromatic moieties are fused directly or indirectly, or a combination thereof as well as non-fused aromatic hydrocarbon rings, fused aromatic hydrocarbon rings.

A substituted or unsubstituted aromatic hydrocarbon cycle may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted terphenyl group, a substituted or unsubstituted quaterphenyl group, a substituted or unsubstituted chrysenyl group, a substituted or unsubstituted triphenylenyl group, a substituted or unsubstituted perylenyl group, a substituted or unsubstituted indenyl group, a combination thereof, or a combined fused ring of the foregoing groups.

As used herein, when a definition is not otherwise provided, “hetero” means including one or more hetero atoms selected from N, O, S, Se, and P.

As used herein, when a definition is not otherwise provided, “heteroaromatic ring” means including at least one hetero atom selected from N, O, S, Se, and P instead of carbon (C) in an aromatic hydrocarbon ring. Two or more heteroaromatic rings may be directly linked through a sigma bond, and two or more heteroaromatic rings may be fused to each other. When the heteroaromatic ring is a fused ring, the entire fused ring or each ring may include 1 to 3 heteroatoms.

Also, as used herein, the polymer may include both an oligomer and a polymer.

Unless otherwise specified in the present specification, the “weight average molecular weight” is measured by dissolving a powder sample in tetrahydrofuran (THF) and then using 1200 series Gel Permeation Chromatography (GPC) of Agilent Technologies (column is Shodex Company LF-804, standard sample is Shodex company polystyrene).

As used herein, the term “or” is not an exclusive term, e.g., “A or B” would include A, B, or A and B.

The hardmask composition according to an embodiment may include a compound including a plurality of aromatic rings or heteroaromatic rings in one molecule to increase a carbon content thereof. Thereby, etch resistance of a hardmask layer formed of the composition may be improved. In an implementation, since the aromatic rings or heteroaromatic rings may include functional groups, solubility of the compound including a plurality of the aromatic rings or the heteroaromatic rings in a solvent may be improved.

In an implementation, the hardmask composition according to an embodiment may include a compound represented by Chemical Formula 1, and a solvent.

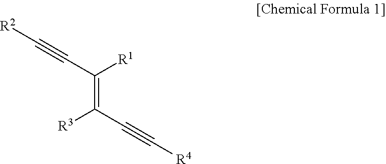

In Chemical Formula 1, R¹ and R³ may each independently be or include, e.g., a substituted or unsubstituted C6 to C30 aromatic hydrocarbon group, a substituted or unsubstituted C3 to C30 heteroaromatic ring group, or a combination thereof, R² and R⁴ may each independently be or include, e.g., a substituted or unsubstituted C6 to C30 aromatic hydrocarbon group, a substituted or unsubstituted C3 to C30 heteroaromatic ring group, a substituted or unsubstituted monovalent C3 to C30 saturated or unsaturated alicyclic hydrocarbon group, or a combination thereof.

The compound represented by Chemical Formula 1 included in the hardmask composition according to an embodiment may include a structure in which two carbon triple bonds are linked to a carbon double bond, which may increase a carbon content in the compound. In an implementation, the compound, may include an aromatic hydrocarbon group, a heteroaromatic ring group, or a saturated or unsaturated alicyclic hydrocarbon group independently linked to the double bond and the triple bond, which may increase the carbon content of the compound.

In an implementation, if the composition including the compound is heat-treated, a carbon ring may be additionally formed by electron transfer within the compound. In an implementation, a carbon content in a hardmask layer formed of the composition may not only be maximized, but also the hardmask layer may exhibit excellent etch resistance.

In Chemical Formula 1, R¹ and R³ may each independently be or include, e.g., a substituted or unsubstituted C6 to C20 aromatic hydrocarbon group, a substituted or unsubstituted C3 to C20 heteroaromatic ring group, or a combination thereof, e.g., a substituted or unsubstituted C6 to C30 aromatic hydrocarbon group, or a substituted or unsubstituted C6 to C20 aromatic hydrocarbon group.

In an implementation, R¹ and R³ of Chemical Formula 1 may be the substituted aromatic hydrocarbon group or heteroaromatic ring groups, wherein the “substituted” may be, e.g., “substituted with a hydroxy group, a substituted or unsubstituted C1 to C4 alkoxy group, a halogen atom, an amino group, or a thiol group, or a combination of,” or “substituted with a hydroxy group, a methoxy group, or an ethoxy group.” If R¹ and R³ are substituted with the above substituents, the solubility of the compound in the solvent may be further improved.

In an embodiment, R¹ and R³ in Chemical Formula 1 may each independently be or include, e.g., substituted or unsubstituted moieties selected from Group 1 and Group 2.

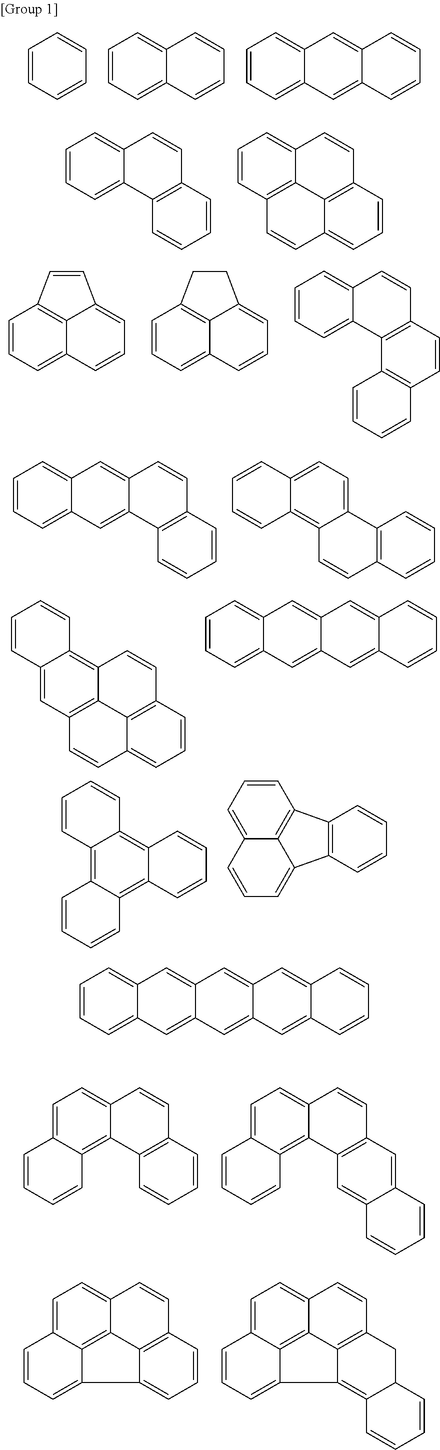

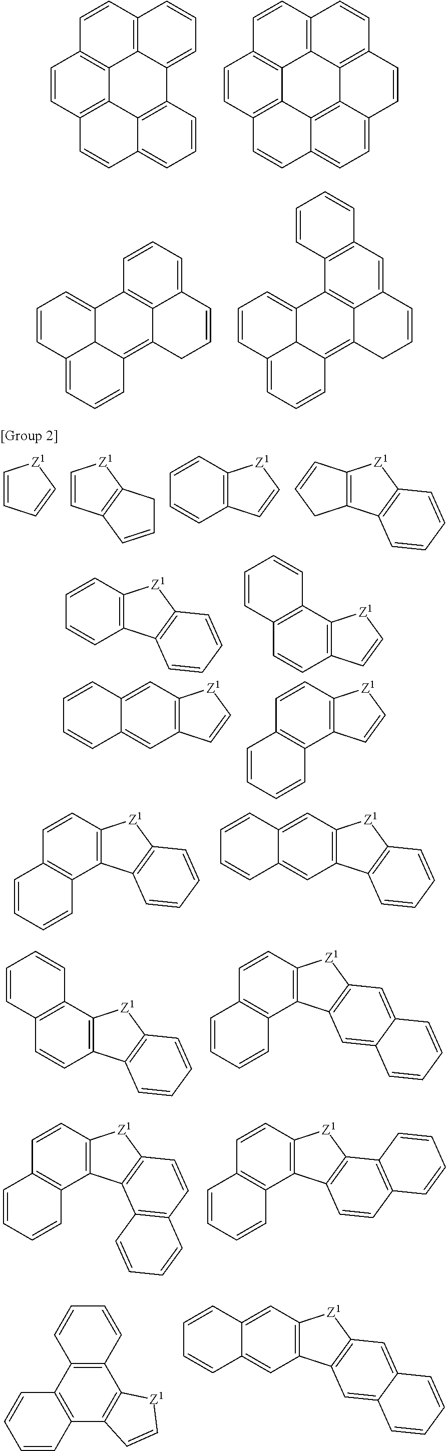

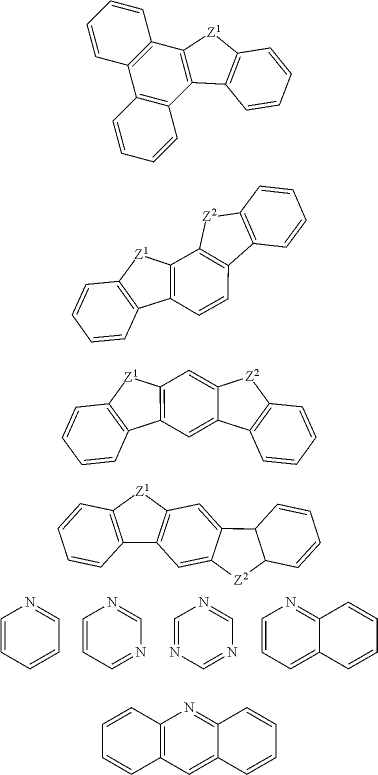

In Group 2, Z¹ and Z² may each independently be or include, e.g., —O—, —S—, —NR^{a}— (wherein, R^{a} may be or include, e.g., hydrogen, deuterium, or a substituted or unsubstituted C1 to C10 alkyl group), or a combination thereof.

In Chemical Formula 1, R² and R⁴ may each independently be or include, e.g., a substituted or unsubstituted C6 to C24 aromatic hydrocarbon group, a substituted or unsubstituted C3 to C24 heteroaromatic ring group, a substituted or unsubstituted monovalent C3 to C24 saturated or unsaturated alicyclic hydrocarbon group, or a combination thereof, e.g., a substituted or unsubstituted C6 to C30 aromatic hydrocarbon group, a substituted or unsubstituted C3 to C30 heteroaromatic ring group, or a combination thereof, or a substituted or unsubstituted C6 to C30 aromatic hydrocarbon group.

In an embodiment, R² and R⁴ in Chemical Formula 1 may each independently be or include, e.g., substituted or unsubstituted moieties selected from Group 1, Group 2, and Group 3.

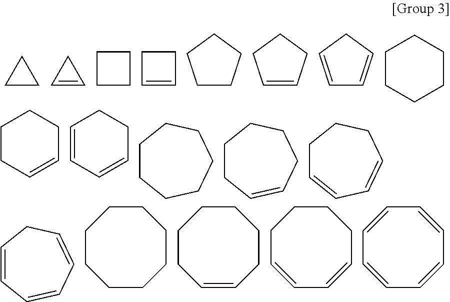

In an implementation, R¹ to R⁴ in Chemical Formula 1 may each independently be or include, e.g., substituted or unsubstituted moieties selected from Group 1-1.

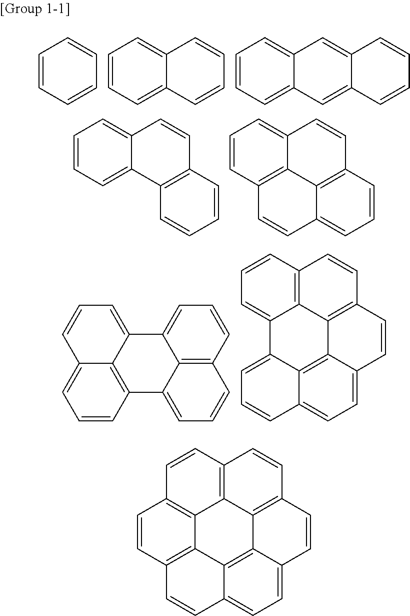

R¹ to R⁴ of Chemical Formula 1 may be different or the same as each other, e.g., two or all of R¹ to R⁴ may be the same. In an implementation, R¹ and R³ among R¹ to R⁴ may be the same, while R² and R⁴ may be the same, wherein Chemical Formula 1 may have a symmetric structure. In an implementation, R¹ and R³ among the R¹ to R⁴ may be different, while R² and R⁴ may be different, wherein Chemical Formula 1 may have an asymmetric structure.

In an implementation, Chemical Formula 1 may be represented by one of the following Chemical Formulae.

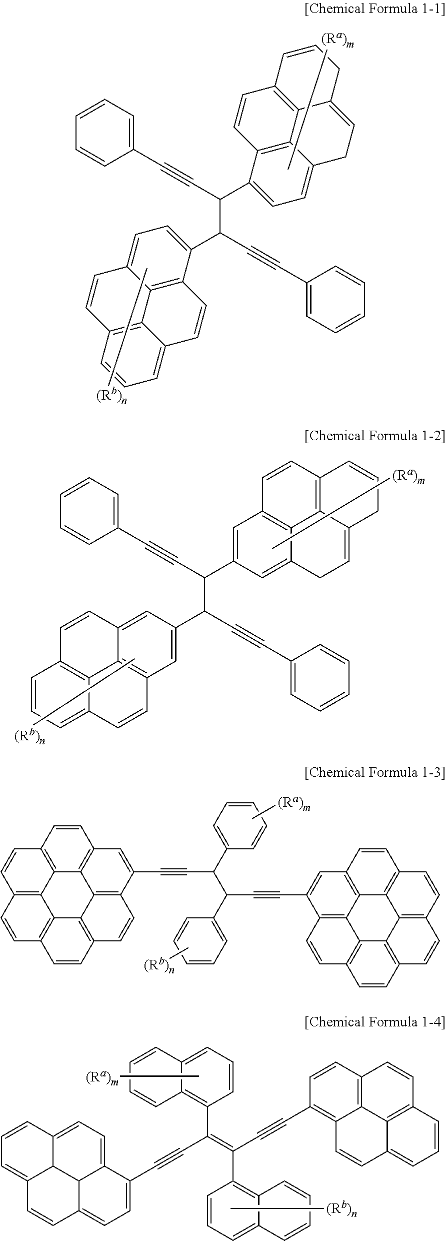

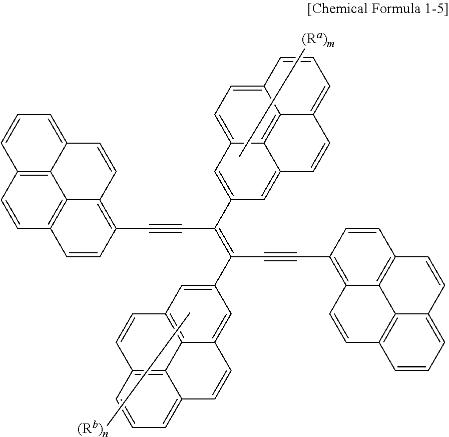

In Chemical Formula 1-1 to Chemical Formula 1-5, R^{a} and R^{b} may each independently be or include, e.g., a hydroxy group, a substituted or unsubstituted C1 to C4 alkoxy group, a halogen atom, an amino group, or thiol group, or a combination thereof, and m and n may each independently be an integer of 0 to 9.

The compound may have a molecular weight of about 300 g/mol to about 5,000 g/mol. In an implementation, it may have a molecular weight of about 300 g/mol to about 4,500 g/mol, about 300 g/mol to about 4,000 g/mol, about 400 g/mol to about 4,500 g/mol, about 400 g/mol to about 4,000 g/mol, about 500 g/mol to about 5,000 g/mol, about 500 g/mol to about 4,500 g/mol, about 500 g/mol to about 4,000 g/mol, or about 500 g/mol to about 3,500 g/mol. By having a molecular weight in the above ranges, the carbon content and solubility in the solvent of the hard mask composition including the above compound may be adjusted and optimized.

The compound may be included in an amount of about 0.1 wt % to about 30 wt % based on the total weight of the hardmask composition. In an implementation, the compound may be included in an amount of about 0.2 wt % to about 30 wt %, e.g., about 0.5 wt % to about 30 wt %, about 1 wt % to about 30 wt %, about 1 wt % to about 25 wt %, or about 1 wt % to about 20 wt %. By including the compound within the above ranges, a thickness, a surface roughness, and a planarization degree of the hardmask may be easily adjusted.

The hardmask composition according to an embodiment may include a solvent, and in an embodiment, the solvent may be, e.g., propylene glycol, propylene glycol diacetate, methoxy propanediol, diethylene glycol, diethylene glycol butyl ether, tri(ethylene glycol) monomethyl ether, propylene glycol monomethyl ether, propylene glycol monomethyl ether acetate, cyclohexanone, ethyl lactate, gamma-butyrolactone, N,N-dimethylformamide, N,N-dimethylacetamide, methylpyrrolidone, methylpyrrolidinone, acetylacetone, ethyl 3-ethoxypropionate, and the like.

The hardmask composition may further include additives such as a surfactant, a crosslinking agent, a thermal acid generator, and a plasticizer.

The surfactant may include, e.g., a fluoroalkyl-based compound, an alkylbenzenesulfonate, an alkylpyridinium salt, polyethylene glycol, a quaternary ammonium salt, and the like.

The crosslinking agent may be, e.g., a melamine, a substituted urea, or a polymer crosslinking agent. In an implementation, it may be a crosslinking agent having at least two crosslinking substituents, e.g., methoxymethylated glycoruryl, butoxymethylated glycoruryl, methoxymethylated melamine, butoxymethylated melamine, methoxymethylated benzoguanamine, butoxy methylated benzoguanamine, methoxymethylated urea, butoxymethylated urea, methoxymethylated thiourea, or butoxymethylated thiourea.

In an implementation, as the crosslinking agent, a crosslinking agent having high heat resistance may be used. The crosslinking agent having high heat resistance may include a compound containing a crosslinking substituent having an aromatic ring (e.g., a benzene ring or a naphthalene ring) in the molecule.

The thermal acid generator may be an acid compound, e.g., p-toluenesulfonic acid, trifluoromethanesulfonic acid, pyridinium p-toluenesulfonic acid, salicylic acid, sulfosalicylic acid, citric acid, benzoic acid, hydroxybenzoic acid, naphthalenecarboxylic acid and/or 2,4,4,6-tetrabromocyclohexadienone, benzointosylate, 2-nitrobenzyltosylate, and other organic sulfonic acid alkyl esters.

According to another embodiment, a hardmask layer including a cured product of the aforementioned hardmask composition may be provided.

Hereinafter, a method of forming patterns using the aforementioned hardmask composition is described.

A method of forming patterns according to an embodiment may include providing a material layer on a substrate, applying a hardmask composition including the aforementioned compound and solvent on the material layer, heat-treating the hardmask composition to form a hardmask layer, forming a photoresist layer on the hardmask layer, exposing and developing the photoresist layer to form a photoresist pattern, selectively removing the hardmask layer using the photoresist pattern to expose a part of the material layer, and etching the exposed part of the material layer.

The substrate may be, e.g., a silicon wafer, a glass substrate, or a polymer substrate. The material layer is a material to be finally patterned, e.g., a metal layer such as an aluminum layer and a copper layer, a semiconductor layer such as a silicon layer, or an insulation layer such as a silicon oxide layer and a silicon nitride layer. The material layer may be formed through a method, e.g., a chemical vapor deposition (CVD) process.

The hardmask composition may be the same as described above, and may be applied by spin-on coating in a form of a solution. Herein, a thickness of the hardmask composition may be, e.g., about 50 Å to about 200,000 Å.

The heat-treating of the hardmask composition may be performed, e.g, at about 100° C. to about 1,000° C. for about 10 seconds to about 1 hour. In an implementation, the heat-treating of the hardmask composition may include a plurality of heat-treating processes, e.g., a first heat-treating process, and a second heat-treating process.

In an embodiment, the heat-treating of the hardmask composition may include, e.g., one heat-treating process performed at about 100° C. to about 1000° C. for about 10 seconds to about 1 hour, and, e.g., the heat-treating may be performed under an atmosphere of air or nitrogen, or an atmosphere having oxygen concentration of 1 wt % or less.

In an implementation, the heat-treating of the hardmask composition may include, e.g., a first heat-treatment process performed at about 100° C. to about 1,000° C., about 100° C. to about 800° C., about 100° C. to about 500° C., or about 150° C. to about 400° C., e.g., for about 30 seconds to about 1 hour, about 30 seconds to about 30 minutes, about 30 seconds to about 10 minutes, or about 30 seconds to about 5 minutes.

In an implementation, the heat-treating of the hardmask composition may include, e.g., a second heat-treatment process performed at about 100° C. to 1,000° C., about 300° C. to about 1,000° C., about 500° C. to about 1,000° C., about 500° C. to about 600° C., e.g., for about 30 seconds to about 1 hour, about 30 seconds to about 30 minutes, about 30 seconds to about 10 minutes, or about 30 seconds to about 5 minutes, consecutively after the first heat-treatment process. In an implementation, the first and second heat-treating process may be performed in an air or nitrogen atmosphere, or may be performed in an atmosphere with an oxygen concentration of 1 wt % or less.

By performing at least one of the steps of heat-treating the hardmask composition at a high temperature of 200° C. or higher, high etch resistance capable of withstanding etching gas and chemical liquid exposed in subsequent processes including the etching process may be exhibited.

In an embodiment, the forming of the hardmask layer may include a UV/Vis curing process or a near IR curing process.

In an embodiment, the forming of the hardmask layer may include at least one of a first heat-treating process, a second heat-treating process, a UV/Vis curing process, or a near IR curing process, or may include two or more processes consecutively.

In an embodiment, the method may further include forming a silicon-containing thin layer on the hardmask layer. The silicon-containing thin layer may be formed of a material, e.g., SiCN, SiOC, SION, SiOCN, SiC, SiO, SiN, or the like.

In an embodiment, the method may further include forming a bottom antireflective coating (BARC) on the silicon-containing thin layer or on the hardmask layer before forming the photoresist layer.

In an embodiment, exposure of the photoresist layer may be performed using, e.g., ArF, KrF, or EUV. After exposure, heat-treating may be performed at about 100° C. to about 700° C.

In an embodiment, the etching process of the exposed part of the material layer may be performed through a dry etching process using an etching gas and the etching gas may be, e.g., N₂/O₂, CHF₃, CF₄, Cl₂, BCl₃, or a mixed gas thereof.

The etched material layer may be formed in a plurality of patterns, and the plurality of patterns may be, e.g., a metal pattern, a semiconductor pattern, an insulation pattern, or the like, or diverse patterns of a semiconductor integrated circuit device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Synthesis of Compound

### Synthesis Example 1

In a 100 mL round-bottomed flask equipped with a stirrer and cooling tube, 1.5 mol (2.33 g) of diethylzinc, 3 mol (3.79 g) of 1-phenylacetylene, and 12 g of toluene were put under a nitrogen atmosphere and then, stirred at 120° C. for 5 hours. After cooling to 0° C., 0.5 mol (1.45 g) of 1-pyrenecarboxaldehyde was slowly added thereto over 20 minutes and then, stirred at room temperature for 12 hours. When the reaction was completed, which was confirmed through thin layer chromatography (TLC), 10 mL of a saturated ammonium chloride aqueous solution was added to an intermediate therefrom and then, extracted with ethyl acetate, and an organic layer therefrom was dried by using sodium sulfate and purified through column chromatography to obtain Compound A represented by Chemical Formula A.

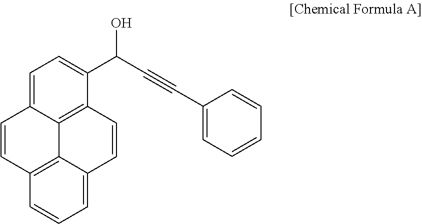 [SMILES: OC(C#Cc1ccccc1)c1ccc2ccc3cccc4ccc1c2c34]

In a 100 mL round-bottomed flask equipped with a stirrer and a cooling tube, 2 mol (2.13 g) of titanium tetrachloride, 3 mol (1.7 g) of triethylamine, and 89 g of dichloromethane were put under a nitrogen atmosphere and then, stirred at 0° C. Subsequently, 2 mol (5.13 g) of Compound A was added thereto and then, stirred at room temperature for 10 hours. When the reaction was completed, 25 g of a saturated chloride ammonium aqueous solution was added thereto and then, stirred for 10 minutes. After separating an organic layer therefrom, an aqueous layer was extracted by using dichloromethane. The organic layer was washed with a saturated sodium chloride aqueous solution and then, treated with sodium sulfate to remove a solvent, and the residues was processed through silica gel column chromatography with hexane to obtain Compound 1 represented by Chemical Formula A-1.

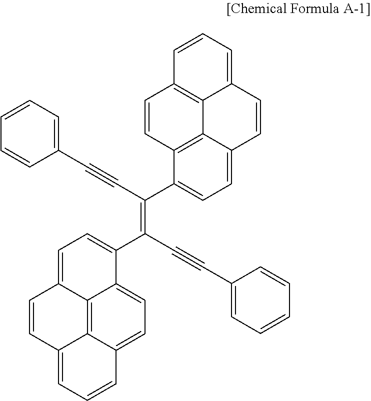 [SMILES: C(#Cc1ccccc1)/C(=C(/C#Cc1ccccc1)c1ccc2ccc3cccc4ccc1c2c34)c1ccc2ccc3cccc4ccc1c2c34]

### Synthesis Example 2

Compound B represented by Chemical Formula B was obtained in the same manner as in Synthesis Example 1 except that 1.55 g of 6-hydroxypyrene-2-carbaldehyde was used instead of the 1-pyrenecarboaldehyde.

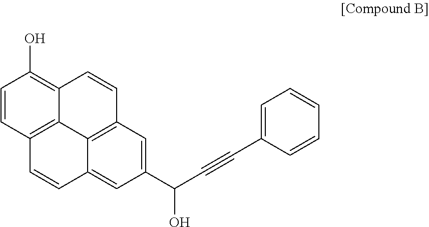 [SMILES: Oc1ccc2ccc3cc(C(O)C#Cc4ccccc4)cc4ccc1c2c34]

Compound 2 represented by Chemical Formula B-1 was obtained in the same manner as in Synthesis Example 1 except that Compound B was used instead of Compound A.

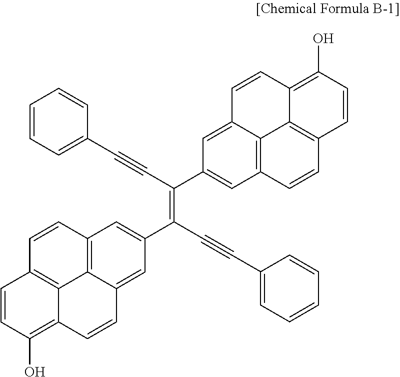 [SMILES: Oc1ccc2ccc3cc(/C(C#Cc4ccccc4)=C(\C#Cc4ccccc4)c4cc5ccc6ccc(O)c7ccc(c4)c5c67)cc4ccc1c2c34]

### Synthesis Example 3

Compound C represented by Chemical Formula C was obtained in the same manner as in Synthesis Example 1 except that 2.07 g of coronenecarboxaldehyde was used instead of the 1-pyrenecarboxaldehyde.

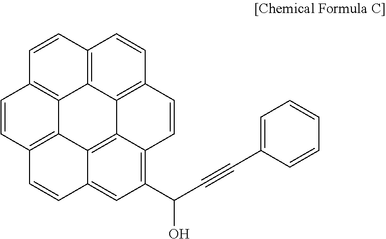 [SMILES: OC(C#Cc1ccccc1)c1cc2ccc3ccc4ccc5ccc6ccc1c1c6c5c4c3c21]

Compound 3 represented by Chemical Formula C-1 was obtained in the same manner as in Synthesis Example 1 except that Compound C was used instead of Compound A.

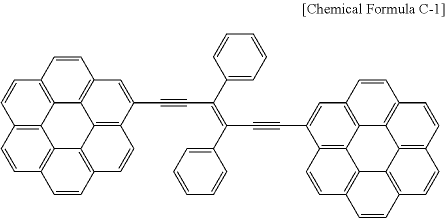 [SMILES: C(#Cc1cc2ccc3ccc4ccc5ccc6ccc1c1c6c5c4c3c21)/C(=C(/C#Cc1cc2ccc3ccc4ccc5ccc6ccc1c1c6c5c4c3c21)c1ccccc1)c1ccccc1]

### Synthesis Example 4

Compound D represented by Chemical Formula D was obtained in the same manner as in Synthesis Example 1 except that 8.54 g of 1-ethynylpyrene instead of the 1-phenylacetylene and 1.08 g of 6-hydroxy-2-naphthaldehyde instead of the 1-pyrenecarboxaldehyde was used.

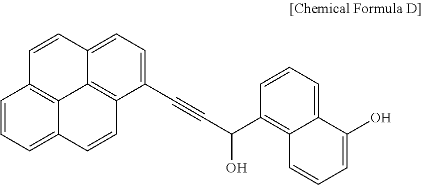 [SMILES: Oc1cccc2c(C(O)C#Cc3ccc4ccc5cccc6ccc3c4c56)cccc12]

Compound 4 represented by Chemical Formula D-1 was obtained in the same manner as in Synthesis Example 1 except that Compound D was used instead of Compound A.

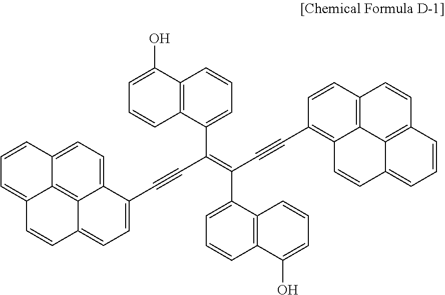 [SMILES: Oc1cccc2c(/C(C#Cc3ccc4ccc5cccc6ccc3c4c56)=C(\C#Cc3ccc4ccc5cccc6ccc3c4c56)c3cccc4c(O)cccc34)cccc12]

### Synthesis Example 5

Compound E represented by Chemical Formula E was obtained in the same manner as in Synthesis Example 1 except that 8.54 g of 1-ethynylpyrene instead of the 1-phenylacetylene and 1.55 g of 6-hydroxy-2-pyrenecarboxaldehyde instead of the 1-pyrenecarboxaldehyde was used.

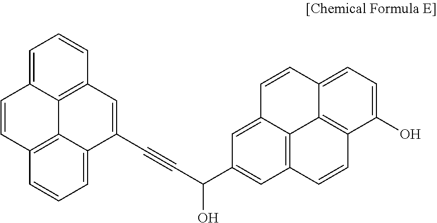 [SMILES: Oc1ccc2ccc3cc(C(O)C#Cc4cc5cccc6ccc7cccc4c7c65)cc4ccc1c2c34]

Compound 5 represented by Chemical Formula E-1 was obtained in the same manner as in Synthesis Example 1 except that Compound E was used instead of Compound A.

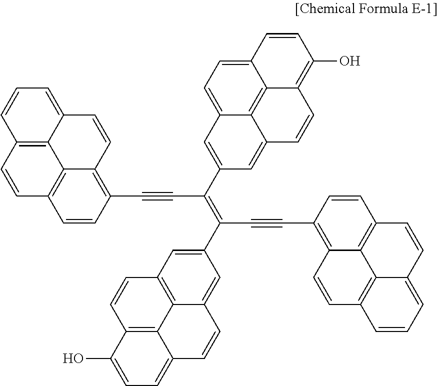 [SMILES: Oc1ccc2ccc3cc(/C(C#Cc4ccc5ccc6cccc7ccc4c5c67)=C(\C#Cc4ccc5ccc6cccc7ccc4c5c67)c4cc5ccc6ccc(O)c7ccc(c4)c5c67)cc4ccc1c2c34]

### Comparative Synthesis Example 1

After putting a 100 mL round-bottomed flask equipped with a stirrer and cooling tube in an ice bath, 0.011 mol (2.44 g) of 1-methoxypyrene, 0.022 mol (5.56 g) of pyrene-1-carbonylchloride, and 32.00 g of 1,2-dichloromethane was put in the round-bottomed flask and then, stirred. Subsequently, 2.87 g of aluminum chloride was added thereto three times at 10-minute intervals under a nitrogen atmosphere. The flask was taken out of the ice bath and then, allowed to stand at room temperature for 10 hours to complete the reaction. After moving the flask to the ice bath again, tetrahydrofuran (THF) and deionized water (DIW) each in a small amount were added to the reaction solution in a dropwise fashion and then, stirred to remove HCl gas. The reaction solution was transferred to a separatory funnel and treated with ethyl acetate and distilled water to remove a catalyst, and an organic layer was separated therefrom and dried under a reduced pressure to remove a solvent. Subsequently, a compound obtained therefrom was dissolved in 40 g of tetrahydrofuran and then, slowly added in a dropwise fashion to 400 mL of normal hexane for precipitation, and precipitates were purified with a filter and dried to obtain 6.02 g of the compound as powder. The obtained powder was dried at 40° C. in a vacuum oven to completely remove the solvent. In a 250 mL round-bottomed flask, the powder, 0.027 mol (11.45 g) of 1-dodecanethiol, 0.036 mol (4.231 g) of potassium hydroxide, and 34.11 g of 1-methyl-2-pyrrolidine were put and then, stirred. A reaction was terminated 8 hours after heating the flask to 90° C. (Mw-675 Da) After cooling to 50° C., 20 g of normal hexane was added thereto and then, stirred for 30 minutes and allowed to strand for 3 hours. After removing a supernatant therefrom, the reaction solution was transferred to a separatory funnel and then, treated with 1,2-dichloromethane, distilled water, and a 5% HCl aqueous solution to remove a catalyst, and when pH becomes 6, an organic layer alone was separated therefrom and dried under a reduced pressure to remove a solvent. Subsequently, a compound obtained therefrom was dissolved in 40 g of tetrahydrofuran and then, slowly added in a dropwise fashion to 400 mL of normal hexane for precipitation, and precipitates were purified with a filter and dried to obtain 4.89 g of the compound as powder. The obtained powder with 19.56 g of tetrahydrofuran was put in a 100 mL round-bottomed flask and stirred in an ice bath. Subsequently, 0.056 mol (2.19 g) of sodium borohydride was dissolved in 19.56 g of deionized water (DIW) in a separate PP bottle and then, added to the flask in three portions. The flask was transferred to an oil bath, stabilized for 10 minutes, and heated to 50° C. After 4 hours, a reaction was completed (Mw-679 Da). After cooling, the reaction solution was transferred to a separatory funnel as in the previous step and then, treated with 1,2-dichloromethane, distilled water, and a 5% HCl aqueous solution to remove a catalyst. A compound obtained therefrom was dissolved in 40 g of tetrahydrofuran and then, slowly added in a dropwise fashion to 400 mL of normal hexane for precipitation, and precipitates were purified with a filter and dried to obtain Comparative Compound 1 represented by Chemical Formula F.

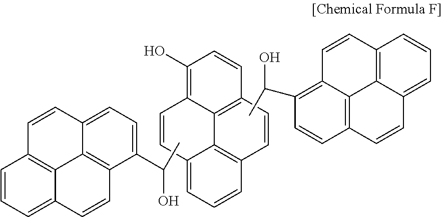

### Comparative Synthesis Example 2

Comparative Compound 2 represented by Chemical Formula G was obtained in the same manner as in Comparative Synthesis Example 1 except that 1,4-benzenedicarbonyl dichloride and pyrene are used instead of the 1-methoxypyrene and the pyrene-1-carbonylchloride.

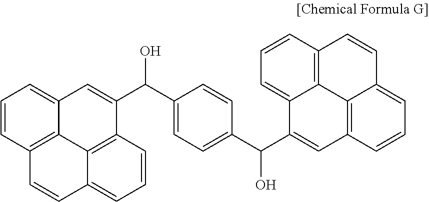 [SMILES: OC(c1ccc(C(O)c2cc3cccc4ccc5cccc2c5c43)cc1)c1cc2cccc3ccc4cccc1c4c32]

### Preparation of Hardmask Composition

### Example 1

A hardmask composition was prepared by dissolving 3.5 g of the compound represented by Chemical Formula A-1 according to Example 1 in 10 g of a mixed solvent of propylene glycolmethyletheracetate and cyclohexanone in a ratio of 7:3 and filtering the solution with a syringe filter.

### Example 2

A hardmask composition was prepared in the same manner as in Example 1 except that the compound represented by Chemical Formula B-1 was used instead of the compound represented by Chemical Formula A-1.

### Example 3

A hardmask composition was prepared in the same manner as in Example 1 except that the compound represented by Chemical Formula C-1 was used instead of the compound represented by Chemical Formula A-1.

### Example 4

A hardmask composition was prepared in the same manner as in Example 1 except that the compound represented by Chemical Formula D-1 was used instead of the compound represented by Chemical Formula A-1.

### Example 5

A hardmask composition was prepared in the same manner as in Example 1 except that the compound represented by Chemical Formula E-1 was used instead of the compound represented by Chemical Formula A-1.

### Comparative Example 1

A hardmask composition was prepared in the same manner as in Example 1 except that the compound represented by Chemical Formula F was used instead of the compound represented by Chemical Formula A-1.

### Comparative Example 2

A hardmask composition was prepared in the same manner as in Example 1 except that the compound represented by Chemical Formula G was used instead of the compound represented by Chemical Formula A-1.

### Evaluation 1: Evaluation of Etch Resistance

Each of the compositions according to the examples and the comparative examples was spin-coated on a silicon wafer and then, baked at 200° C. for 60 seconds to form a 1,500 Å-thick hardmask layer. On each of the hardmask layers, a photoresist for ArF was coated at 110° C. for 60 seconds, exposed to light by using an exposure equipment (XT:1450G, NA 0.93, ASML), and developed in TMAH (a 2.38 wt % aqueous solution) to form a 60 nm line and space pattern. The photoresist pattern was further cured at 110° C. for 60 seconds and then, used with a CHF₃/CF₄ mixed gas to dry-etch the hardmask layer for 20 seconds, and then, a cross-section thereof was examined with FE-SEM to measure an etching rate and thus evaluate etch resistance to halogen plasma. The etch resistance was evaluated according to the following criteria, and the results are shown in Table 1.

### <Evaluation Criteria for Etch Resistance>

⊚: Etching rate is less than 10 Å/sec
O: Etching rate is greater than or equal to 10 Å/sec and less than 11 Å/sec
Δ: Etching rate is greater than or equal to 11 Å/sec and less than 12 Å/sec
X: Etching rate is greater than or equal to 12 Å/sec

TABLE 1

| | Etch resistance |
|---|---|
| Example 1 | ⊚ |
| Example 2 | ⊚ |
| Example 3 | ⊚ |
| Example 4 | ⊚ |
| Example 5 | ⊚ |
| Comparative Example 1 | ◯ |
| Comparative Example 2 | Δ |

Referring to Table 1, each of the hardmask layers formed of the hardmask compositions of Examples 1 to 5 exhibited a lower etching rate of less than 10 Å/sec than 10 Å/sec or more of the hardmask layers of the hardmask compositions of Comparative Examples 1 and 2. In other words, each of the hardmask layers of the hardmask compositions of Examples 1 to 5 exhibited excellent etch resistance, compared with those of the comparative examples.

By way of summation and review, the typical lithographic technique may include providing a material layer on a semiconductor substrate; coating a photoresist layer thereon; exposing and developing the same to provide a photoresist pattern; and etching a material layer using the photoresist pattern as a mask.

Nowadays, according to the small size of the pattern to be formed, it may be difficult to provide a fine pattern having an excellent profile use only the above-mentioned typical lithographic technique. Accordingly, an auxiliary layer, called a hardmask layer, may be formed between the material layer and the photoresist layer to help provide a fine pattern.

There is a constant trend in a semiconductor industry to reduce a size of chips, and in order to cope with this demand, a line width of a resist may be patterned to have several tens of nanometers through lithography. A height of the resist may be limited in order to maintain the line width of the resist pattern, but the resist may not have sufficient resistance in the etching process. In order to compensate for this, an auxiliary layer, which is called a hardmask layer, may be used between a material layer to be etched and a photoresist layer. This hardmask layer may serve as an interlayer that transfers a fine pattern of the photoresist layer through selective etching and thus may be required to have sufficient etch resistance to withstand the etching process during the pattern transfer.

A conventional hardmask layer may be formed in a chemical or physical deposition method and may have a problem of low economic efficiency due to large-scale equipment and a high process cost. Accordingly, a spin-coating technique for forming a hardmask layer has recently been developed. The spin-coating technique may be an easier process to conduct than the conventional method, and a hardmask layer formed therefrom may exhibit much more excellent gap-fill characteristics and planarization characteristics, but there is a tendency that etch resistance required for the hardmask layer is deteriorated. Accordingly, a hardmask composition may be required to apply to the spin-coating technique and to secure equivalent etch resistance to that of the hardmask layer formed in the chemical or physical deposition method.

Accordingly, in order to improve the etch resistance of a hardmask layer, research on maximizing a carbon content of the hardmask composition is being actively made. However, as a carbon content of a compound included in the hardmask composition is maximized, solubility of the compound in a solvent may decrease. Accordingly, a hardmask composition may be required by maximizing a carbon content of a compound included therein to improve etch resistance of a hardmask layer formed thereof without deteriorating solubility of the compound in a solvent should not be deteriorated.

The hardmask composition according to the embodiment may have excellent solubility in a solvent and thus may be effectively applied to the hardmask layer. Additionally, the hardmask layer formed from the hardmask composition according to the embodiment may secure excellent etch resistance.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A hardmask composition, comprising:
a solvent; and
a compound represented by Chemical Formula 1: 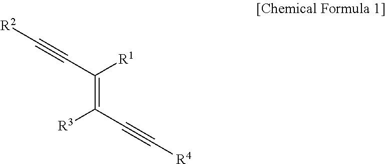
wherein, in Chemical Formula 1,
R¹ and R³ are each independently a substituted or unsubstituted C6 to C30 aromatic hydrocarbon group, a substituted or unsubstituted C3 to C30 heteroaromatic ring group, or a combination thereof, and
R² and R⁴ are each independently a substituted or unsubstituted C6 to C30 aromatic hydrocarbon group, a substituted or unsubstituted C3 to C30 heteroaromatic ring group, a substituted or unsubstituted monovalent C3 to C30 saturated or unsaturated alicyclic hydrocarbon group, or a combination thereof.

2. The hardmask composition as claimed in claim 1, wherein:
R¹ and R³ are each independently a substituted or unsubstituted C6 to C20 aromatic hydrocarbon group, a substituted or unsubstituted C3 to C20 heteroaromatic ring group, or a combination thereof, and
R² and R⁴ are each independently a substituted or unsubstituted C6 to C24 aromatic hydrocarbon group, a substituted or unsubstituted C3 to C24 heteroaromatic ring group, a substituted or unsubstituted monovalent C3 to C24 saturated or unsaturated alicyclic hydrocarbon group, or a combination thereof.

3. The hardmask composition as claimed in claim 1, wherein:
R¹ and R³ are each independently a substituted or unsubstituted C6 to C30 aromatic hydrocarbon group, and
R² and R⁴ are each independently a substituted or unsubstituted C6 to C30 aromatic hydrocarbon group, a substituted or unsubstituted C3 to C30 heteroaromatic ring group, or a combination thereof.

4. The hardmask composition as claimed in claim 1, wherein:
R¹ and R³ are each independently a substituted or unsubstituted C6 to C20 aromatic hydrocarbon group, and
R² and R⁴ are each independently a substituted or unsubstituted C6 to C30 aromatic hydrocarbon group.

5. The hardmask composition as claimed in claim 1, wherein:
R¹ and R³ each independently include a substituted or unsubstituted moiety of Group 1 or Group 2, and
R² and R⁴ each independently include a substituted or unsubstituted moiety of Group 1 to Group 3: 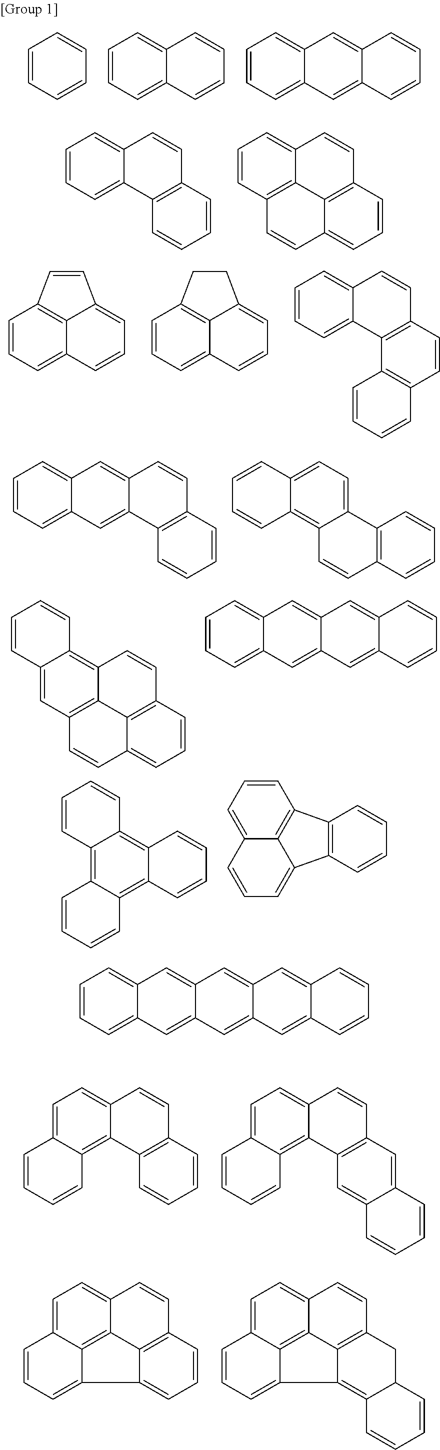 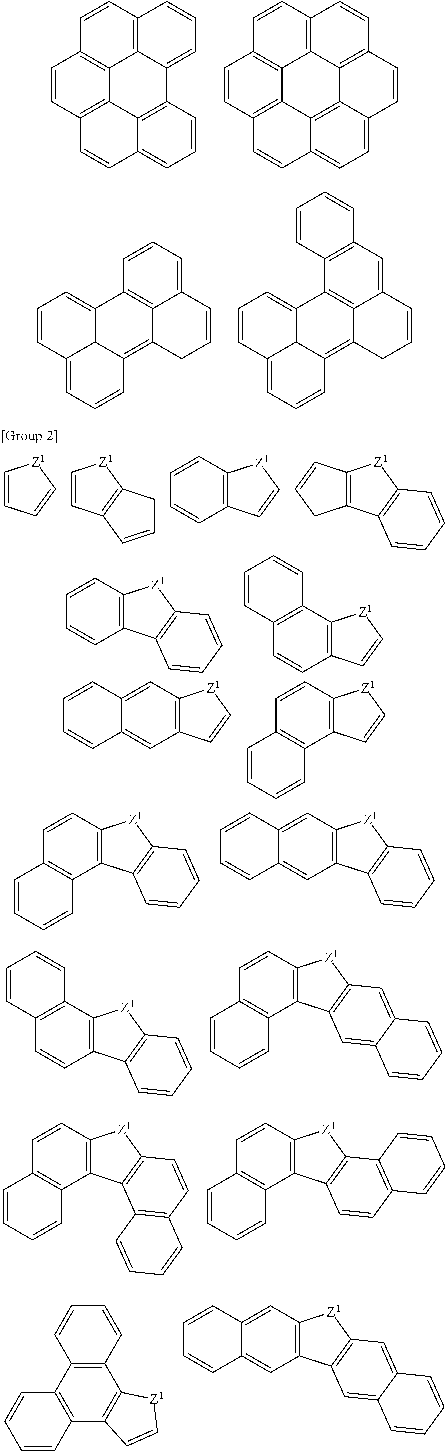 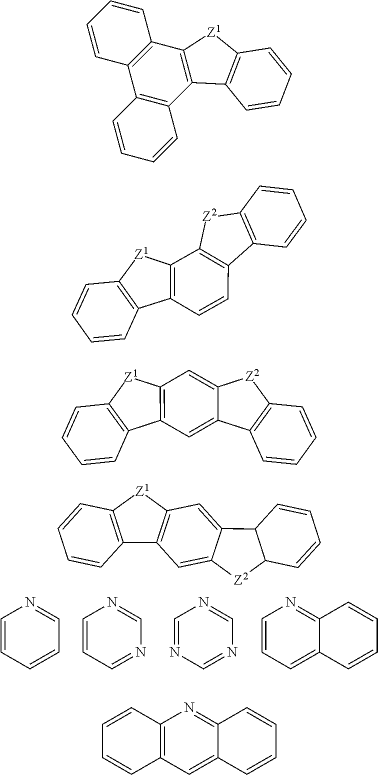
in Group 2,
Z¹ and Z² are each independently —O—, —S—, —NR^{a}—, or a combination thereof, and
R^{a} is hydrogen, deuterium, or a substituted or unsubstituted C1 to C10 alkyl group.

6. The hardmask composition as claimed in claim 1, wherein R¹ to R⁴ each independently include a substituted or unsubstituted moiety of Group 1-1:

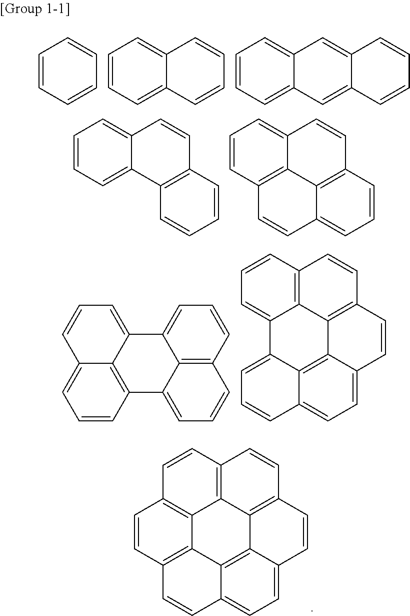

7. The hardmask composition as claimed in claim 1, wherein the compound has a symmetrical structure.

8. The hardmask composition as claimed in claim 1, wherein:
the compound is represented by one of the following Chemical Formulae: 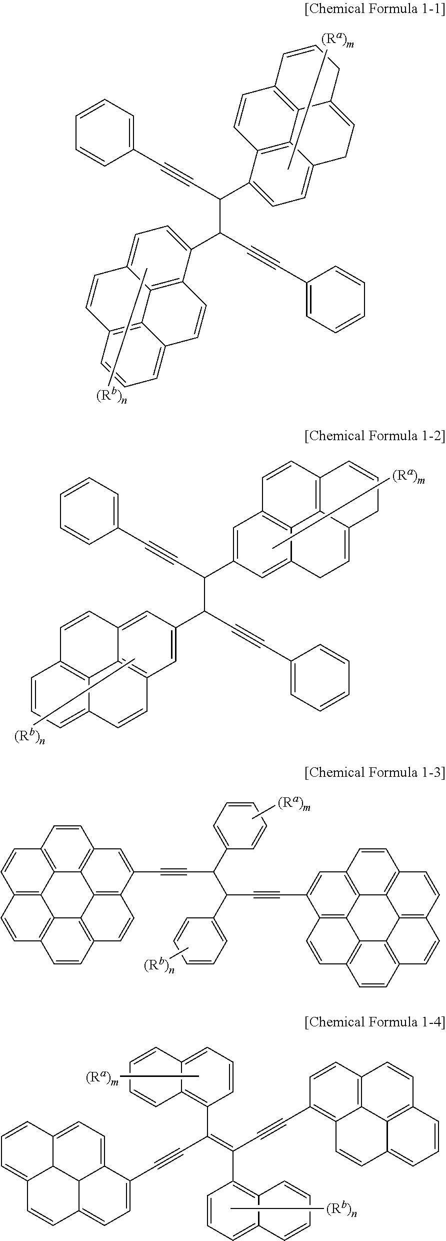 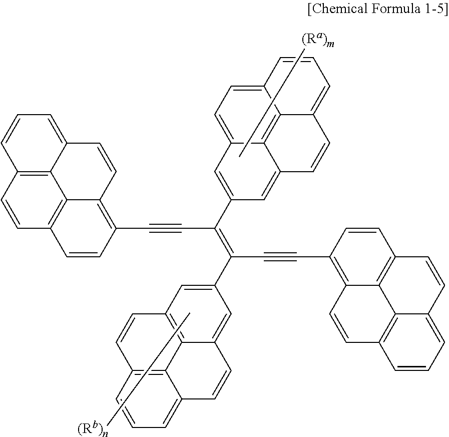
in Chemical Formula 1-1 to Chemical Formula 1-5,
R^{a} and R^{b} are each independently a hydroxy group, a substituted or unsubstituted C1 to C4 alkoxy group, a halogen atom, an amino group, a thiol group, or a combination thereof, and
m and n are each independently an integer of 0 to 9.

9. The hardmask composition as claimed in claim 1, wherein the compound has a molecular weight of about 300 g/mol to about 5,000 g/mol.

10. The hardmask composition as claimed in claim 1, wherein the compound is included in an amount of about 0.1 wt % to about 30 wt % based on a total weight of the hardmask composition.

11. The hardmask composition as claimed in claim 1, wherein the solvent is propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, methylpyrrolidinone, acetylacetone, or ethyl 3-ethoxypropionate.

12. A hardmask layer comprising a cured product of the aforementioned hardmask composition as claimed in claim 1.

13. A method of forming patterns, the method comprising:
providing a material layer on a substrate;
applying the hardmask composition as claimed in claim 1 on the material layer;
heat-treating the hardmask composition to form a hardmask layer;
forming a photoresist layer on the hardmask layer;
exposing and developing the photoresist layer to form a photoresist pattern;
selectively removing the hardmask layer using the photoresist pattern to expose a portion of the material layer; and
etching an exposed part of the material layer.

14. The method as claimed in claim 13, wherein the hardmask composition is heat-treated at about 100° C. to about 1,000° C.

15. The method as claimed in claim 13, wherein the hardmask composition is heat-treated a first time and a second time.

16. The method as claimed in claim 15, wherein the hardmask composition is heat-treated the first time at about 100° C. to about 1,000° C. for about 30 seconds to about 1 hour.

17. The method as claimed in claim 15, wherein the hardmask composition is heat-treated the second time at about 100° C. to about 1,000° C. for about 30 seconds to about 1 hour.

18. The method as claimed in claim 15, wherein the hardmask composition is heat-treated the first or second time at about 200° C. or higher.

19. The method as claimed in claim 13, wherein the hardmask composition has a thickness of about 50 Å to about 200,000 Å.

20. The method as claimed in claim 13, wherein the hard mask composition is applied by spin-on coating.
